(19) United States
(12) Patent Application Publication
(10) Pub. No.: US 2025/0173863 A1
(43) Pub. Date: May 29, 2025

(54) METHODS AND SYSTEMS RELATING TO ARTIFICIAL INTELLIGENCE FOR EARLY RECOGNITION OF RARE SKIN DISEASES

(71) Applicant: Northwestern University, Evanston, IL (US)

(72) Inventors: Antonia REIMER-TASCHENBRECKER, Evanston, IL (US); Amy PALLER, Evanston, IL (US); Abel N. KHO, Evanston, IL (US)

(21) Appl. No.: 18/929,268

(22) Filed: Oct. 28, 2024

Related U.S. Application Data
(60) Provisional application No. 63/593,845, filed on Oct. 27, 2023.

(51) Int. Cl. G06T 7/00; A61B 5/00; G06V 10/764; G06V 10/774; G06V 10/82; G16H 50/20

(52) U.S. Cl. CPC G06T 7/0012; A61B 5/445; A61B 5/7267; G06V 10/764; G06V 10/774; G06V 10/82; G16H 50/20; G06T 2207/20081; G06T 2207/20084; G06T 2207/30088; G06T 2207/30096; G06V 2201/03

(57) ABSTRACT

The present disclosure provides methods and systems to artificial intelligence for early recognition of rare skin diseases. In particular, the present disclosure provides methods and systems for early recognition of squamous cell carcinoma in epidermolysis bullosa and methods and systems for treating and/or preventing SCCs.

### CROSS-REFERENCE TO RELATED APPLICATIONS

This application claims the benefit of U.S. Provisional Patent Application No. 63/593,845, filed on Oct. 27, 2023, which is incorporated by reference herein.

### FIELD

The present disclosure provides methods and systems related to artificial intelligence for early recognition of rare skin diseases. In particular, the present disclosure provides methods and systems for early recognition of squamous cell carcinoma in epidermolysis bullosa and methods and systems for treating and/or preventing SCCs.

### BACKGROUND

Recessive dystrophic epidermolysis bullosa is a rare hereditary skin condition. The skin is fragile, minor injuries lead to blisters and wounds, and patients suffer from frequent pain and infections. As a severe complication, patients with recessive dystrophic epidermolysis bullosa (RDEB) develop skin cancer, particularly squamous cell carcinoma (SCC). SCCs in RDEB occur at a young age, are aggressive, and there is no available non-invasive tool for detection of SCCs. Early discovery is key, but recognizing SCC is difficult. Tools are needed to analyze photographs of RDEB skin and to identify SCCs.

### SUMMARY

Embodiments of the present disclosure include a computer-based method comprising a) imaging a lesion on human skin selected from the group consisting of: (i) epidermolysis bullosa (EB) skin; (ii) non-EB skin; (iii) squamous cell carcinoma (SCC) skin; (iv) non-SCC skin; (v) EB SCC skin; and (vi) non-EB SCC skin; to provide an imaged human skin; b) annotating the one or more lesion of the imaged human skin; c) assessing a potential for a disease of the imaged human skin; and d) choosing a therapy for the imaged human skin.

In some embodiments, the method further comprises a smartphone application.

In some embodiments, the assessing a potential for a disease of the imaged human skin comprises collection of electronic health data.

Embodiments of the present disclosure also include a computer-based system comprising a) a one or more imaging device; b) a one or more communication component) a one or more memory component; d) a one or more information processing component configured to analyze data; e) a protocol component; wherein the computer-based system (i) assesses a potential for a disease of the imaged human skin; (ii) chooses a therapy for the imaged human skin; and (iii) performs a one or more action on the imaged human skin.

In some embodiments, the one or more communication component comprises an input device and an output device.

In some embodiments, the protocol component comprises one or more protocols for: (i) imaging a processed human skin to provide an imaged human skin; (ii) annotating a one or more lesion of the imaged human skin; (iii) assessing a potential for malignancy of the imaged human skin; and (iv) choosing a therapy for the imaged human skin.

In some embodiments, the one or more information processing component or the protocol component alters protocols based on responses from a software.

In some embodiments, the assessing a potential for a disease of the imaged human skin comprises collection of electronic health data.

Embodiments of the present disclosure also include a method of treating a subject who is suffering from skin condition, the method comprising providing the subject with the computer-based system comprising a) a one or more imaging device; b) a one or more communication component) a one or more memory component; d) a one or more information processing component configured to analyze data; e) a protocol component; wherein the computer-based system assesses a potential for malignancy of the imaged human skin and performs actions on the imaged human skin.

In some embodiments, provided herein are methods of developing an artificial intelligence (AI) tool for distinguishing between two medical conditions, the method comprising: (a) training the AI tool on a large-scale hierarchical image database to generate an initially trained model with robust non-specific feature-detection capabilities; (b) adapting the initially trained model to the medical conditions by transfer learning to binary classification of images of the two medical conditions to generate model trained on the two medical conditions; (c) combining output of the del trained on the two medical conditions with patient clinical data to generate a combined model; and (d) training a Random Forest-based meta-learner classifier with the combined model to generate the AI tool for distinguishing between two medical conditions. In some embodiments, the large-scale hierarchical image database is not specific to images of the two medical conditions. In some embodiments, the image is obtained by a non-medical professional. In some embodiments, methods further comprise applying augmentation techniques to expand diversity of the images of the two medical conditions used to trail the AI tool. In some embodiments, the augmentation techniques comprise transformations of the images of the two medical conditions used to trail the AI tool. In some embodiments, transformations of the images comprise one or more of rotations, flips, scaling, and color adjustments. In some embodiments, the two medical conditions are skin conditions. In some embodiments, the two medical conditions can be differentiated visually. In some embodiments, the two medical conditions are (1) squamous cell carcinoma (SCC) lesion in a subject suffering from epidermolysis bullosa and (2) non-SCC lesion in a subject suffering from epidermolysis bullosa. In some embodiments, the subject suffers from recessive dystrophic epidermolysis bullosa (RDEB). In some embodiments, provided herein are methods of determining whether a subject suffers from a first or second medical condition, the method comprising: (a) obtaining an image of a region of the subject displaying a symptom of the medical condition; (b) providing the image to an AI tool developed by the method herein; and (c) determining whether the subject suffers from the first or second condition.

In some embodiments, provided herein are methods of developing an artificial intelligence (AI) tool for distinguishing between (1) a squamous cell carcinoma (SCC) lesion in a subject suffering from epidermolysis bullosa and (2) a non-SCC lesion in a subject suffering from epidermolysis bullosa: (a) training the AI tool on a large-scale hierarchical image database not limited to SCC lesions and/or subjects suffering from EB to generate an initially trained model with robust non-specific feature-detection capabilities; (b) adapting the initially trained model to the medical conditions by transfer learning to binary classification of images of SCC lesions and non-SCC lesions in subject suffering from EB to generate specific model trained on SCC lesions and non-SCC lesions in subject suffering from EB; (c) combining output of the specific model with patient clinical data to generate a combined model; and (d) training a Random Forest-based meta-learner classifier with the combined model to generate the AI tool for distinguishing between SCC lesions and non-SCC lesions in subject suffering from EB. In some embodiments, the subject suffers from recessive dystrophic epidermolysis bullosa (RDEB).

In some embodiments, provided herein are methods of distinguishing between an SCC lesion and a non-SCC lesion in subject suffering from EB, the method comprising: (a) obtaining an image of the lesion; (b) providing the image to an AI tool developed by the method herein; (c) determining whether the subject suffers from an SCC lesion or a non-SCC lesion. In some embodiments, methods further comprise treating the subject for an SCC lesion or a non-SCC lesion based on the outcome of step (c).

In some embodiments, provided herein are methods of determining whether a lesion on a subject suffering from epidermolysis bullosa (EB) is a squamous cell carcinoma (SCC) lesion or a non-SCC lesion, the method comprising: (a) obtaining an image of the lesion; (b) providing the image to an AI tool trained to distinguish between SCC lesions and non-SCC lesions on a subject suffering from EB; and (c) determining whether the lesion is an SCC lesion of a non-SCC lesion. In some embodiments, method further comprise administering appropriate treatment for an SCC lesion or non-SCC lesion.

In some embodiments, provided herein is software (e.g., an app) or a device (e.g., a computer, tablet, phone, etc.) containing a AI tool described herein. In some embodiments, provided herein is software (e.g., an app) or a device (e.g., a computer, tablet, phone, etc.) for performing a method herein.

### BRIEF DESCRIPTION OF THE DRAWINGS

FIG. 1: Diagnosis of RDEB-SCCs-RDEB skin images showing many of the same features.

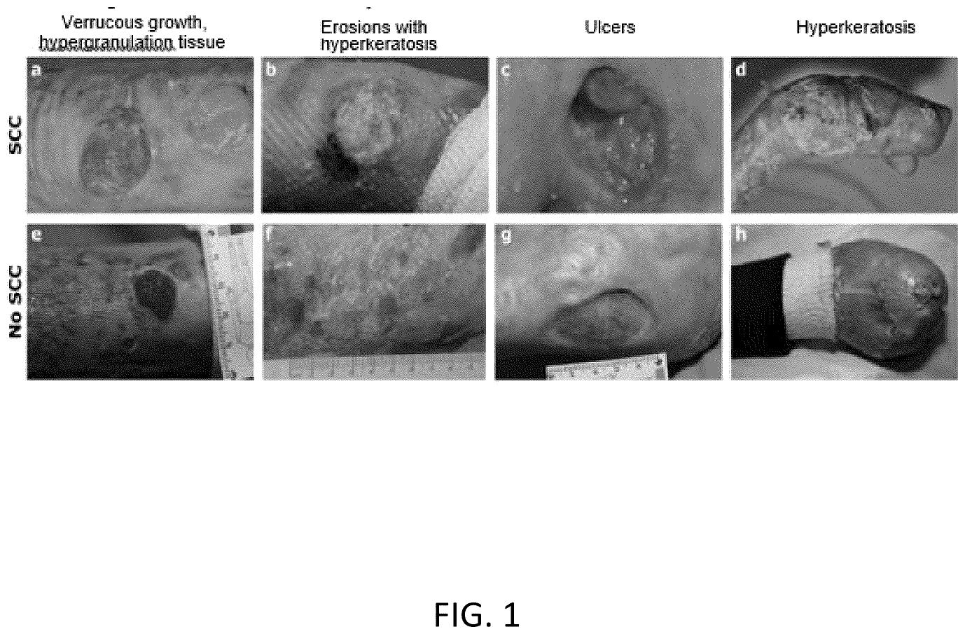

FIG. 2: Example of manual image annotation and labels.

FIG. 3: Flow-chart of an example set-up for image curation, meta-learning module, and test module.

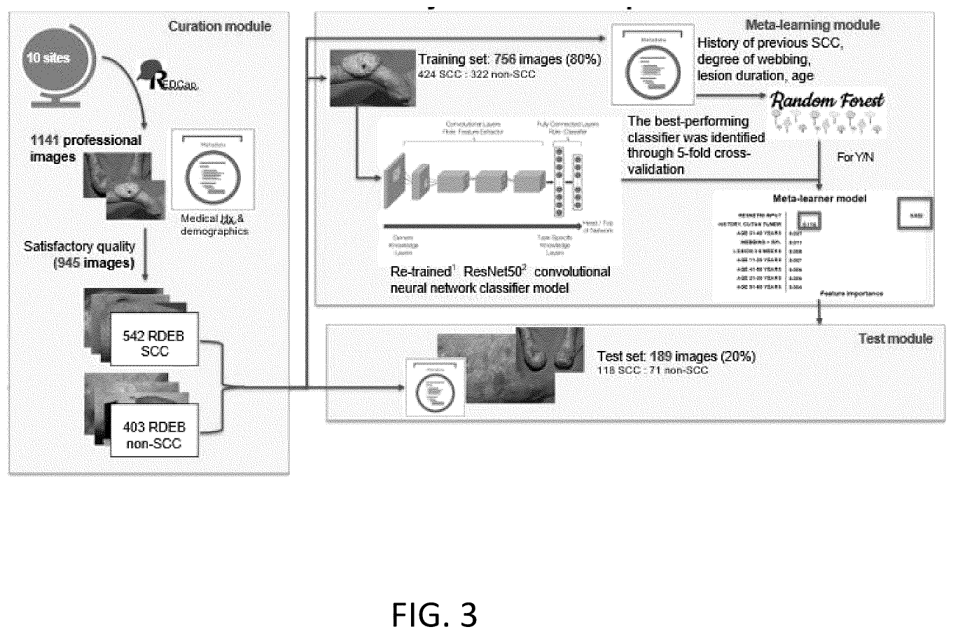

FIG. 4: Test module results. Summary of meta-learner model performance.

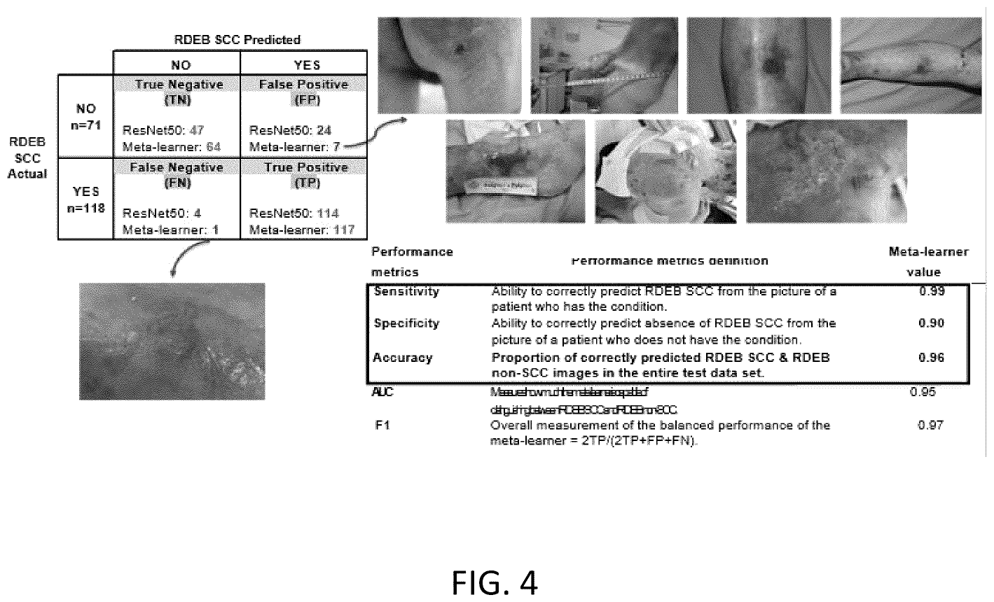

FIG. 5: Example of point-by-point image annotation (freeform) of features and areas suspicious for or known to have SCC.

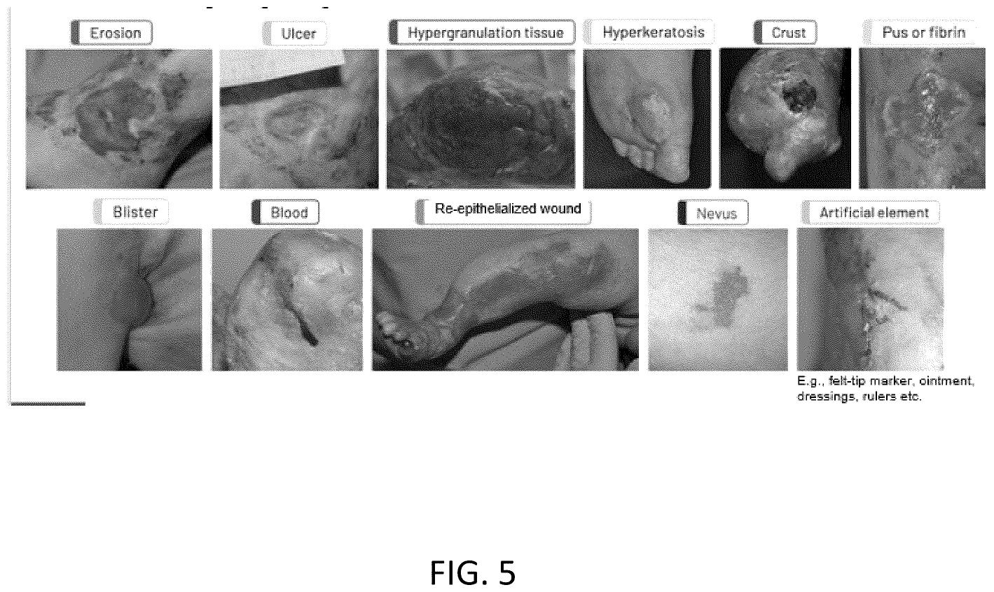

FIG. 6: Example of image annotation of features known to have SCC.

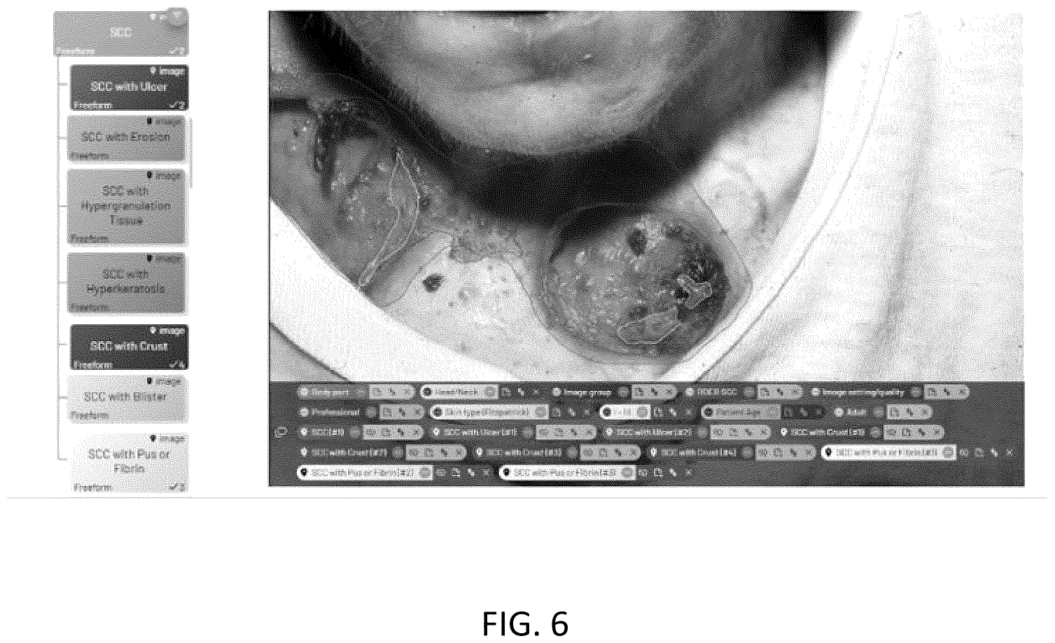

FIG. 7: Flow-chart of an example set-up for image curation, meta-learning module, validation and refinement module, test module, and smartphone application.

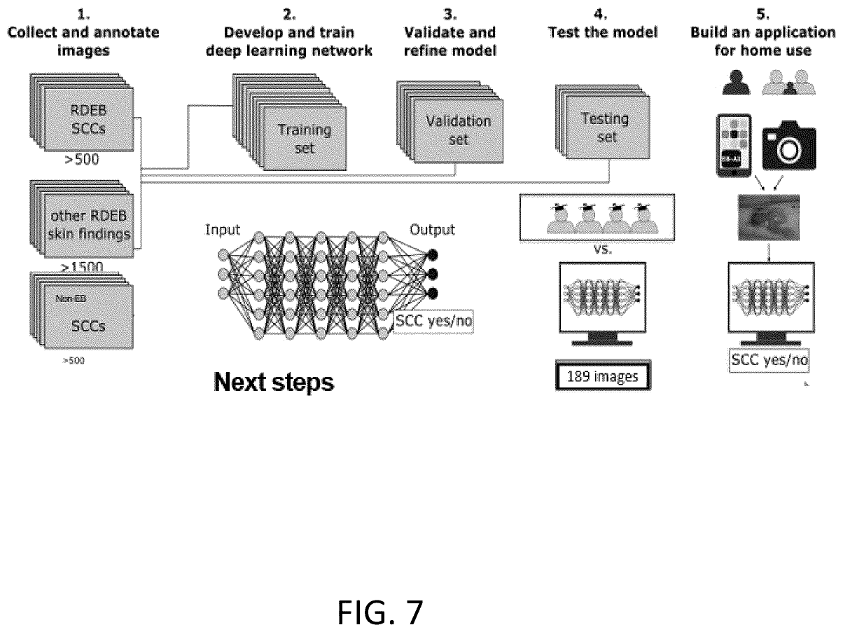

FIG. 8: Example of misdiagnosis due to weakness of image recognition in deep learning.

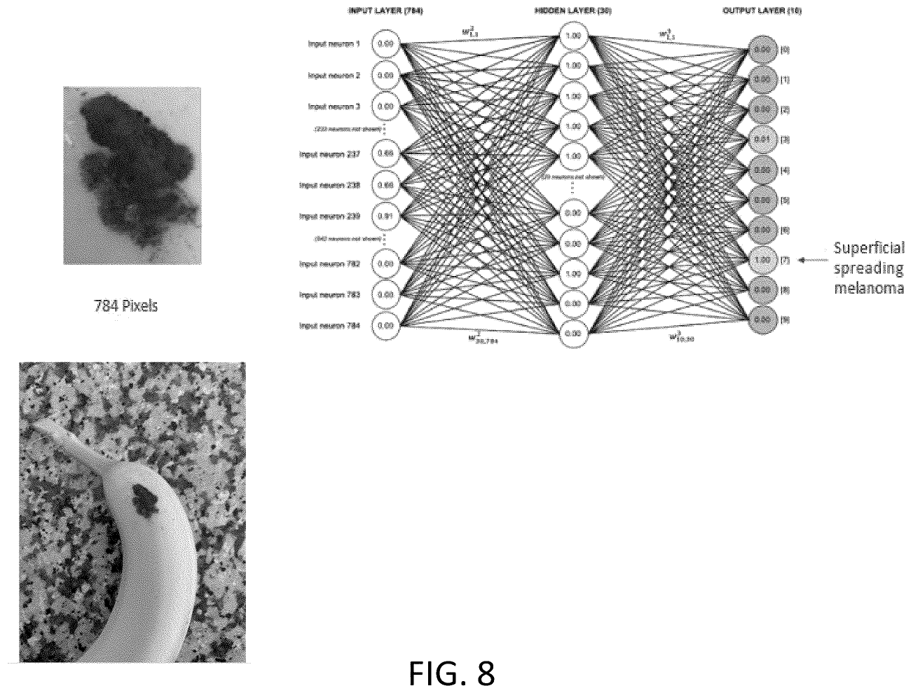

FIG. 9: Comparison of AUC plots between meta learner models and clinician majority votes across test set subsets A to E.

FIG. 10: Depiction of RDEB Skin and AI insigth: Left panel shows original images of RDEB skin with marked SCC areas (circled in yellow). Right panel presents ResNet50 heatmaps with normalized CAM contours. Each image includes MD majority vote diagnostic and AI predictions with corresponding probabilities and 95% CIs.

FIG. 11: Data processing workflow for AI training and testing alongside international clinician evaluations.

FIG. 12: Mean SHAP values for each image in the test set, derived from the ResNet50 model trained on original RDEB images, highlight the model's interpretability by showing the relative importance of different image regions in predicting outcomes.

FIG. 13: Depiction of RDEB Skin and AI insight: Left panel shows images of RDEB skin without backgrounds with marked SCC areas (circled in yellow). Right panel presents ResNet50 heatmaps with normalized CAM contours. Each image includes MD majority vote diagnostic and AI predictions with corresponding probabilities and 95% CIs.

FIG. 14: ROC AUC curves depicting diagnostic performance for individual MD reviewers (Groups A-E) and the aggregated majority vote. The curves highlight comparative sensitivity and specificity, with majority voting showing overall improved performance.

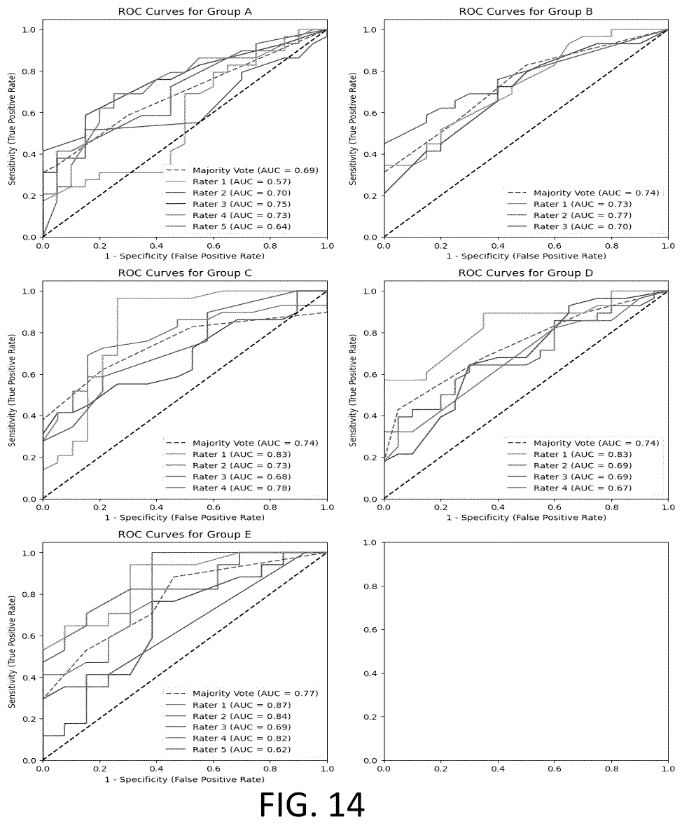

FIG. 15: Feature importance as it is implemented in ResNet50-based meta learner model.

FIG. 16: An exemplary comparative illustration presents an original annotated image of a foot affected by RDEB SCC, featuring contours demonstrating SCC, erosion), and re-epithelialized wound on the left. On the right, a Grad-CAM visualization derived from a ResNet-50 classifier highlights the specific areas within the RDEB SCC that are recommended for biopsy by a clinician to confirm SCC presence.

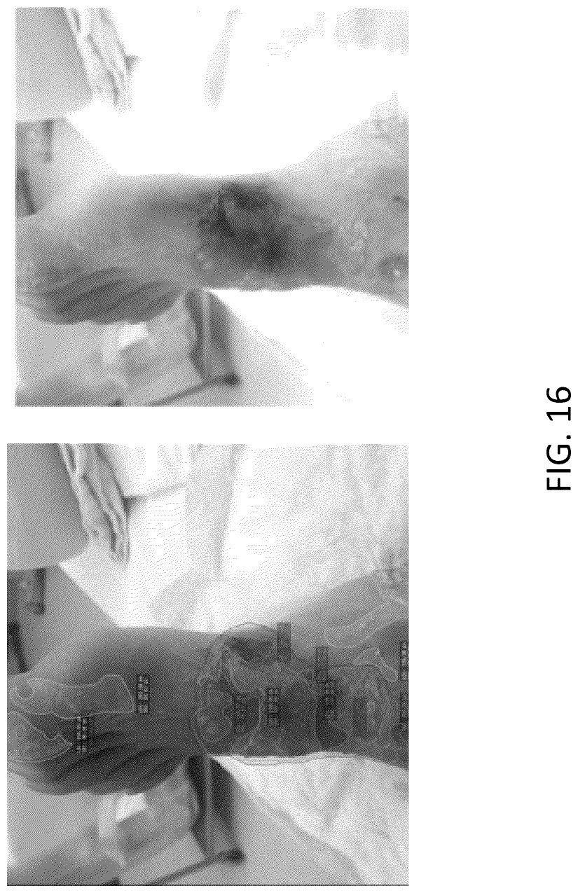

### DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The terms “administration of” and “administering” a composition as used herein refers to providing a composition of the present disclosure to a subject in need of treatment (e.g., antiviral treatment). The compositions of the present disclosure may be administered by oral, parenteral (e.g., intramuscular, intraperitoneal, intravenous, ICV, intracisternal injection or infusion, subcutaneous injection, nebulization, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

The term “artificial intelligence” as used herein refers to Computer performing tasks usually requiring human intelligence (ex. object recognition and complex decision-making).

The term “natural language processing” as used herein refers to processing text or spoken language to make information accessible to computer algorithms.

The term “machine learning” as used herein refers to computers learning from data provided (ex. data provided with human-labelled training data and/or with detectable labels) to gather insights and make predictions (ex. an algorithm clusters data according to its intrinsic features).

As used herein, the term “transfer learning” refer to a machine learning techniques that use pre-trained models to improve performance on a related task. In transfer learning, a model is pre-trained on one task and then fine-tuned for a related task. The model's components are reused to create a new model for the related task.

As used herein the term “random forest analysis” refers to a computational method based using multiple different decision trees to compute the overall most predicted class (the mode). In a specific application herein, the mode will be either RDEB-SCC or RDEB-non-SCC. The class predicted by the majority is selected as the predicted class for the sample.

The term “convoluted neural networks” for images and deep learning as used herein refers to how the nervous system processes information with multiple layers of interconnecting nodes where each performs calculations based on input from other connected nodes (ex. if multiple hidden layers, then deep neural networks).

The term “machine learning stages” as used herein refers to (a) preprocessing (ex., find missing values, resolve inconsistencies, restructure data), (b) splitting data into a training set (ex., generating an algorithm), repeating to improve performance, and using a smaller test set; and (c) choosing ML algorithm(s) based on the problem and data (usually multiple), choosing the best performance model, and validating on test set of unseen data.

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, macaque, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### DETAILED DESCRIPTION

The present disclosure provides methods and systems related to artificial intelligence for early recognition of rare skin diseases. In particular, the present disclosure provides methods and systems for early recognition of squamous cell carcinoma in epidermolysis bullosa and methods and systems for treating and/or preventing SCCs.

Epidermolysis bullosa is a group of rare genodermatoses affecting approximately 500,000 individuals globally, characterized by skin fragility and blistering. Recessive dystrophic epidermolysis bullosa (RDEB) is a severe subtype often complicated by squamous cell carcinoma (SCC), the leading cause of death in these patients. RDEB-SCC diagnosis is challenging, as symptoms often resemble other RDEB-related skin changes. Additionally, patient reluctance for full-body examination and biopsies complicates timely and accurate diagnosis. The aggressive nature of RDEB-SCCs with local destruction and metastases severely limits effective treatment options, emphasizing the critical need for early detection.

Current diagnostic challenges are compounded by the scarcity of dermatologists specialized in genodermatoses like RDEB, posing significant hurdles for routine and specialized care. Residual networks have shown proficiency in identifying skin cancers, such as melanoma, at a level comparable to expert dermatologists. Earlier AI developments have focused on dermoscopic images of “normal” skin, but applying these to macroscopic images of complex, rare diseases like RDEB is unprecedented. Adapting AI for RDEB has the potential to aid physicians in early cancer detection, streamlining the care pathway. By democratizing access to expert-level diagnostic capabilities, AI also addresses health inequalities arising from geographic and resource disparities.

Rare diseases such as RDEB present unique challenges in healthcare, primarily due to the scarcity of expertise and resources needed to diagnose and treat these conditions effectively. With less than 12% of machine learning studies on rare diseases evaluating their algorithms externally or against human expertise, there is a pressing need to improve the access and quality of diagnostics worldwide. AI applications for rare diseases often fail to transition from theoretical models to clinical practice. This gap is primarily due to the lack of external validation in many diagnostic tools designed for rare diseases, a challenge compounded by the difficulty in obtaining and sharing rare disease datasets without breaching patient privacy and involving patient advocacy groups. By conducting external validation using a separate test set and comparing AI performance against RDEB clinical experts, the AI tools herein overcome limitations that previous tools have faced.

Transfer learning has emerged as a key strategy in dealing with the small and imbalanced datasets typical of rare diseases. It mitigates bias and overfitting, ensuring the AI models are robust and generalizable. The choice of leveraging transfer learning contributes to ongoing advancements in improving diagnostic accuracy and availability for patients with rare diseases through innovative technologies. In some embodiments, the AI-based RDEB-SCC diagnostic tool developed herein currently serves Fitzpatrick's skin types I-III within specific clinical photography settings. Embodiments within the scope herein include a wider variety of skin types and amateur-obtained images.

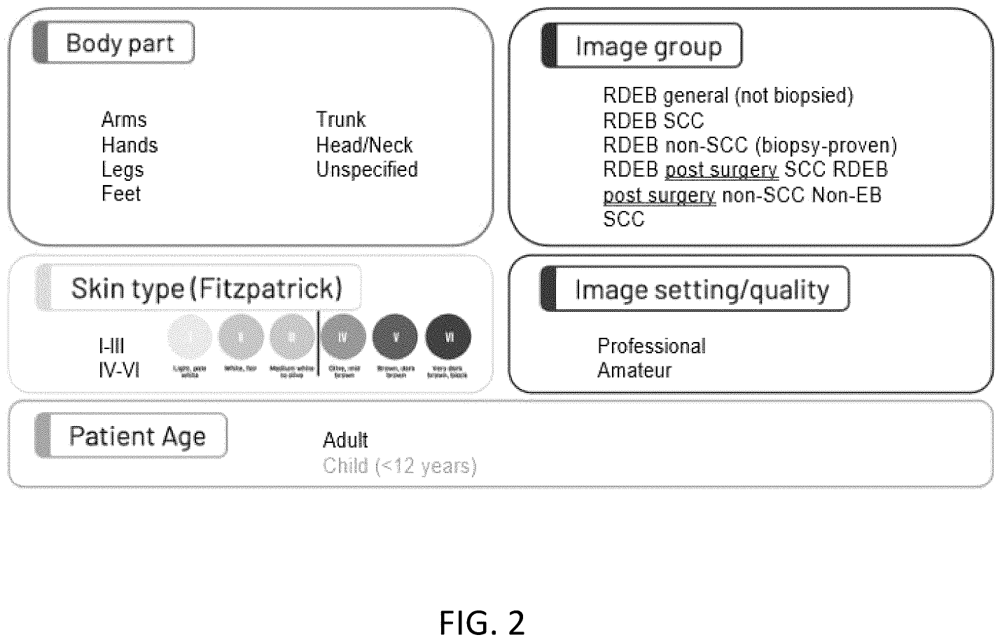

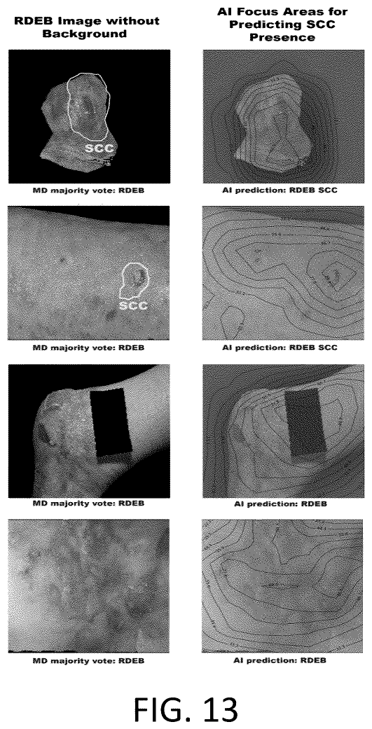

In certain embodiments, the models developed for diagnostic purposes herein utilize both complete images (FIG. 2) and those with background elements removed (FIG. 13) to concentrate solely on relevant skin features. This approach increases diagnostic accuracy by standardizing image evaluations, reducing variability caused by different environments and equipment. It also enhances computational efficiency and response times while minimizing privacy risks by omitting personal background details from images. These tools are particularly useful for remote dermatological assessments, improving healthcare access in underserved areas. Training AI on skin-only images also simplifies learning and improves generalization across various skin types.

In some embodiments, provided herein are AI meta-learners, leveraging Random Forest and Residual Network algorithms, that matches the diagnostic accuracy of international specialists in detecting SCC lesions in RDEB patients. The tools herein combine clinical images and patient data, enhancing healthcare delivery and broadening access to specialized oncology and rare disease expertise.

Embodiments of present disclosure provide methods and systems related to artificial intelligence for early recognition of rare skin diseases. In particular, the present disclosure provides methods and systems for early recognition of squamous cell carcinoma in epidermolysis bullosa and methods and systems for treating and/or preventing SCCs. Section headings as used in this section and the entire disclosure herein are merely for organizational purposes and are not intended to be limiting.

Embodiments of the present disclosure include a computer-based method comprising a) imaging a processed skin (ex., animal skin, human skin (ex. diseased human skin (ex. atopic dermatitis, cold sores, dry skin, psoriasis, vitiligo, contact dermatitis, rosacea, melasma, pemphigus, acne, poison ivy, poison oak, epidermolysis bullosa (EB), squamous cell carcinoma))) to provide an imaged human skin (ex., by whole slide imaging with a scanner or camera); b) annotating a one or more lesion of the imaged human skin (ex., manually or automatically annotating using labels and classification); c) assessing a potential for a disease of the imaged human skin (ex., rare skin diseases (ex., RDEB SCCs, Argyria, Erythropoietic Protoporphyria, Harlequin Ichthyosis, Elastoderma, Interstitial Granulomatous Dermatitis, Pemphigus, Acral Peeling Skin Syndrom)); and d) choosing a therapy for the imaged human skin.

In some embodiments, the method further comprises a application (ex., a computer application configured to a phone, a mobile device, a small computer, or a large computer).

In some embodiments, the assessing a potential for a disease of the imaged human skin comprises collection of electronic health data (ex., patient identifiers, operative notes, demographics, diagnoses, medications, procedures, laboratory data, vital signs, utilization of medical care, cost of medical care, digital health records (ex. all data hosted on a physician's own servers, storage of data from the physician to a third party, remote systems).

Embodiments of the present disclosure also include a method of treating a subject who is suffering from skin condition, the method comprising providing the subject with the computer-based system comprising a) a one or more imaging device; b) a one or more communication component) a one or more memory component; d) a one or more information processing component configured to analyze data; e) a protocol component; wherein the computer-based system assesses a potential for disease of the imaged human skin and performs actions on the imaged human skin.

Embodiments of the present disclosure also include a computer-based system comprising a) a one or more imaging device; b) a one or more communication component) a one or more memory component; d) a one or more information processing component configured to analyze data; e) a protocol component; wherein the computer-based system (i) assesses a potential for a disease of the imaged human skin; (ii) chooses a therapy for the imaged human skin; and (iii) performs a one or more action on the imaged human skin.

In some embodiments, the one or more communication component comprises an input device and an output device.

In some embodiments, the protocol component comprises one or more protocols for: (i) imaging a processed human skin to provide an imaged human skin; (ii) annotating a one or more lesion of the imaged human skin; (iii) assessing a potential for malignancy of the imaged human skin; and (iv) choosing a therapy for the imaged human skin.

In some embodiments, the one or more information processing component or the protocol component alters protocols based on responses from a software.

In some embodiments, the assessing a potential for a disease of the imaged human skin comprises collection of electronic health data.

In some embodiments, method herein comprise treating an SCC lesion in a subject suffering from RDEB (an RDEB-SCC lesion). Method of treating an SCC lesion include surgical removal of the lesion (e.g., Mohs surgery, shave excision, standard excision, etc.), radiation therapy, electrodesiccation and curettage, topical chemotherapy treatment (e.g., fluorouracil), etc.

### EXPERIMENTAL

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Using artificial intelligence (AI) to train a computer to recognize SCC on EB skin. Several international reference centers were joined to amass a large number of photographs of RDEB SCCs and RDEB skin. The photographs are used to train an AI tool to take and analyze photographs of RDEB skin and to identify RDEB-SCCs.

The AI tool is capable of use at clinical centers and at home and will aid clinicians in deciding whether to take a biopsy and which skin area to biopsy. The AI tool is also useful for patients and families between clinic visits, enabling them to capture cutaneous images for review at the visit without going through a time-consuming and painful full bandage change in the doctor's office. Image exchange between patients and their physician is valuable for patients with difficult access to medical expertise and those who are hesitant to allow full skin examination at each visit, often because of pain or fear of biopsies.

Sizable EB populations are merged to amass a large cohort of patients and their images. A joint, anonymized data organization system (using REDCap and SharePoint) is created between the cooperating centers to establish a strong data pipeline. The globally collected sets of photographs of i) RDEB-SCCs and ii) other skin findings in RDEB (e.g., wounds and hyperkeratosis) are used to create a development set and a validation set. A total of i)>500 RDEB-SCC pictures and ii)>1500 pictures of other RDEB skin findings is compiled.

The image classification model is also trained by transfer learning, using images of head and neck cutaneous SCCs in non-EB patients. These share certain molecular characteristics of RDEB-SCC and, although different in course, location, and age of affected individuals, are similar enough in appearance that the classifier can be trained first on that to identify likely SCC and then be applied to RDEB-SCCs. A large image set of >3000 head and neck cutaneous SCCs is compiled from general dermatology, skin surgery, and surgical oncology units. To further enhance the model performance, demographic and clinical features, in addition to image data, is included.

A deep learning network is trained on the development set to recognize RDEB-SCCs and to distinguish them from other RDEB skin findings. Model development and refinement is performed in cooperation with a multi-disciplinary team of informatics experts and computer science individuals. Initial models are developed using convolutional neural networks (CNNs).

To validate the model and to assess its accuracy, the performance of the deep learning network is compared against that of a group of international EB experts. These experts are recruited and include at least 30 individuals. Experts are asked to indicate their experience with EB in years (0-2 years, 2-5 years, >5 years, >10 years). A validation set consisting of 50 images not used in the training phase is created. Participants and the deep learning network are asked for a dichotomous diagnosis (“RDEB-SCC” or “other RDEB skin finding”) on the images of the validation set. Analysis employs descriptive statistics and two-sided t-tests assesses the difference between the performances of the experts and the AI application. The significance level is set at P<0.05.

A smartphone application (app) is created that incorporates the deep learning network intended for use both by physicians at clinical centers as well as by individuals affected by RDEB and their families at home. For the latter purpose, families with teenagers and adults with RDEB are contacted via the patient organization DEBRA International (at least 10 patients/families) to test the app and provide feedback on ease of use, information exchange, and quality of images.

### Example 2

In patients with recessive dystrophic epidermolysis bullosa (RDEB), distinguishing squamous cell carcinoma (SCC) lesions from other skin changes is challenging even for experienced dermatologists, yet early detection improves survival outcomes. Experiments were conducted during development of embodiments herein to develop and validate an artificial intelligence (AI) tool to effectively detect carcinoma in patients with RDEB using images and clinical data.

A retrospective diagnostic study analyzed routinely collected patient data from 11 international dermatology sites, using deep learning models trained, fine-tuned, and cross-validated on 741 biopsy-confirmed macroscopic photographs of RDEB-associated SCCs and non-SCCs. These models, enhanced with patient clinical data through Random Forest models, were evaluated against extra 181 biopsy-confirmed images. The AI's diagnostic accuracy was compared to both individual and consensus assessments from 21 international EB-expert physicians across five datasets, each containing 30-49 images.

Primary outcomes measured diagnostic accuracy of meta-learner models, individual clinicians, and dermatologist consensus through the specificity, sensitivity, and area under the curve (AUC) compared to biopsy-confirmed diagnoses. Secondary outcomes assessed diagnostic agreement among 21 international dermatologists and AI models, using Fleiss Kappa for clinicians and Cohen's Kappa for AI versus clinician consensus. Another secondary outcome assessed the diagnostic accuracy of meta-learner models trained on images with backgrounds removed.

Clinician diagnostic agreement was fair-to-moderate (Fleiss Kappa=0.33±0.12), with AUC values ranging from 0.57 to 0.87; the majority dermatologist vote produced an average AUC of 0.71±0.04, comparable to AI performance. Meta-learner models based on ResNet152 and ResNet50 showed AUCs of 0.77±0.14 and 0.71±0.06 on smaller clinician-evaluated datasets, and 0.76 and 0.81 on an expanded set of 181 images, respectively. Models designed for images with the background removed showed comparable diagnostic accuracy when tested on a dataset of 181 images. The exemplary AI accurately distinguished carcinoma from other lesions in RDEB skin images, matching dermatologist consensus and outperforming individual clinicians. This tool enhances diagnostic precision and accessibility, enables expert-level detection in remote areas, and streamlines continuous cancer monitoring.

### Methods

### Participants

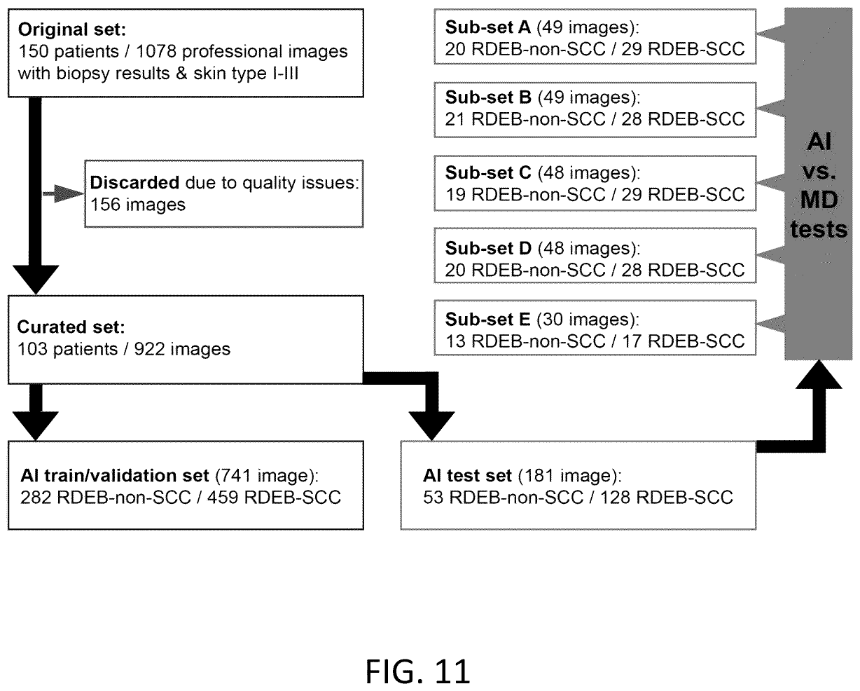

A total of 150 patients diagnosed with RDEB were included in the diagnostic model development. Eligibility criteria required participants to be aged 11 years and older, have Fitzpatrick skin types I-III (pale white to darker white skin), and biopsy- or expert-confirmed SCC presence or absence (FIG. 11).

### Data Preparation

Before analysis, all clinical data and biopsy-confirmed lesion photographs (n=1078) were fully anonymized. 156 images deemed of poor quality by at least two dermatologists, hindering accurate diagnostic assessments, were excluded from the dataset (FIG. 11). Diagnostic-quality images were meticulously curated into subsets: a training/validation set comprising 741 images and a testing set with 181 images. The dataset included multiple images of the same lesion from different angles or similar lesions on the same individual. To preserve data integrity, it was manually verified that these sets remained separate between the training and test phases. Sample size split was determined by lesion variability data and the need for representative training data. The test set balanced representativeness with computational feasibility, encompassing diverse lesion features. To evaluate the accuracy of carcinoma detection by AI versus clinicians, the test set images were divided into five subsets: A (49 images), B (49 images), C (48 images), D (48 images), E (30 images). This arrangement prevented experts from reviewing images originating from their own center.

For all models, images were converted from DICOM to JPG format and standardized to a resolution of 224×224 pixels for consistency. The initial model development and clinician evaluation used JPG images with original backgrounds. A subset of models was additionally trained on images without background; for this, background elements were removed using the Segment Anything Model with subsequent manual validation.

Patient characteristics with significant missing data were excluded, and for those retained, missingness indicators were used. This method avoids imputation and maintains data integrity, as missing values, like lesion location in conditions unrelated to webbing, are inherently informative and not random. Patient information was categorized into binary variables based on age groups, degree of webbing, lesion duration, history of cutaneous SCC, and lesion locations (Table 1).

TABLE 1
Characteristics of Patients in Training and Test Image Sets

| | Images with | |
|---|---|---|
| Feature | RDEB-non-SCC | RDEB-SCC |
| Total, N | 335.00 | 587.00 |
| Age, years | | |
| 11-20 | 75 (22.4%) | 79 (13.5%) |
| 21-30 | 141 (42.1%) | 194 (33%) |
| 31-40 | 80 (23.9%) | 241 (41.1%) |
| 41-50 | 18 (5.4%) | 58 (9.9%) |
| 51-60 | 21 (6.3%) | 15 (2.6%) |
| Sex = Female | 166 (49.6%) | 197 (33.6%) |
| Time since lesion onset, weeks | | |
| <3 | 18 (5.4%) | 5 (0.9%) |
| 5 Mar. | 23 (6.9%) | 51 (8.7%) |
| 12 Jun. | 15 (4.5%) | 24 (4.1%) |
| ≥6 | 174 (51.9%) | 317 (54%) |
| >12 | 159 (47.5%) | 293 (49.9%) |
| NI | 120 (35.8%) | 214 (36.5%) |
| Lesion location | | |
| Hand | 41 (12.2%) | 275 (46.8%) |
| Foot | 71 (21.2%) | 105 (17.9%) |
| Head/neck | 4 (1.2%) | 9 (1.5%) |
| Leg | 43 (12.8%) | 90 (15.3%) |
| Trunk/buttock | 104 (31%) | 28 (4.8%) |
| Arm | 72 (21.5%) | 80 (13.6%) |
| Digital deformity | | |
| Normal hands/no fusion | 16 (4.8%) | 2 (0.3%) |
| 25% | 13 (3.9%) | 4 (0.7%) |
| 50% | 15 (4.5%) | 21 (3.6%) |
| 75% | 26 (7.8%) | 8 (1.4%) |
| 100% | 263 (78.5%) | 475 (80.9%) |
| Milia and/or scarring | 2 (0.6%) | 1 (0.2%) |
| Amputated | 0 (0%) | 44 (7.5%) |
| ≥50% | 306 (91.3%) | 549 (93.5%) |
| ≥75% | 291 (86.9%) | 528 (89.9%) |
| NI | 0 (0%) | 32 (5.5%) |
| No history of cutaneous tumor | 236 (70.4%) | 89 (15.2%) |

### AI Diagnostics

ResNet50 and ResNet152 from the Residual Network family were used as base architectures for detecting SCC from images. These models were initially trained on a large dataset featuring over 14 million images across more than 20,000 classes, which equipped them with robust feature-detection capabilities. Utilizing transfer learning, the team adapted these models for SCC identification in RDEB lesional skin by replacing the original top layer with a new one designed for binary classification into RDEB-SCC and RDEB-non-SCC, incorporating dropout and a dense output layer to enhance generalization and prevent overfitting. To further improve robustness, data augmentation techniques such as random flipping, zooming, and rotation were applied.

The initial layers of the models were frozen to maintain the integrity of the learned features, focusing training on fine-tuning the new layers over 30 epochs using a batch size of 16 images and a learning rate of 0.001, with one-fifth of the data reserved for validation. The Adam optimizer was used for efficient training, and model checkpoints were saved based on the highest validation F1 scores. The training leveraged 5-fold cross-validation, producing five well-performing models. Each model generated an output labeled either as RDEB-SCC or RDEB-non-SCC. Probabilities from these models were averaged to create an ensemble, enhancing prediction reliability. This ensemble output, combined with patient clinical data, was used to train a Random Forest-based meta-learner classifier. This hybrid approach integrates the robust feature extraction of residual models with the ensemble decision-making of Random Forests, optimizing the model's diagnostic performance.

During the meta-learner training, the optimal tree count for the Random Forest was identified by evaluating out-of-bag accuracy over various counts. Recursive Feature Elimination was then used to iteratively refine the feature set (Table 6). The data sets exhibited class imbalances, with ratios of RDEB-SCC to RDEB-non-SCC of 1.63 and 2.42 in the training and testing sets, respectively. To mitigate bias towards the majority class and enhance fairness, the model automatically adjusted the weights inversely to the class frequencies. Further model optimization involved a comprehensive grid search across multiple hyperparameters to fine-tune the settings for improved prediction accuracy. The best parameters were determined through 5-fold cross-validation, ensuring model performance across varied data subsets. The final meta-learner model, trained with optimized parameters, reliably demonstrated its effectiveness through key performance metrics on the withheld test set. The final outputs of the meta-learner models include predicted probabilities of the positive class and their confidence intervals, calculated assuming a normal distribution. For each prediction, the standard deviation is computed from the probability's inherent variance. A Z-score for the 95% confidence level determines the margin of error, which defines the lower and upper bounds of the confidence interval around each predicted probability.

TABLE 6
Feature importance derived from the residual model, alongside mean absolute SHAP values,
highlights the key predictors in residual models trained on original RDEB images. The table
presents the contribution of each feature to the model's predictions, offering insights
into the underlying patterns influencing the detection of SCC in patients with RDEB.

| | Meta learner | | | | | |
|---|---|---|---|---|---|---|
| | ResNet50 - based | | | ResNet152 - based | | |
| | | Mean absolute | | | Mean absolute | |
| | | SHAP value | | | SHAP value | |
| | | RDEB- | RDEB- | | RDEB- | RDEB- |
| Feature | Importance | non-SCC | SCC | Importance | non-SCC | Scc |
| No history of | 0.226 | 0.498 | 0.503 | 0.221 | 0.496 | 0.462 |
| cutaneous tumor | | | | | | |
| ResNet output | 0.125 | 0.481 | 0.512 | 0.223 | 0.509 | 0.433 |
| Lesion location arm | 0.067 | 0.498 | 0.492 | 0.052 | 0.493 | 0.537 |
| Lesion location hand | 0.064 | 0.519 | 0.517 | 0.054 | 0.480 | 0.521 |
| Lesion location | 0.057 | 0.510 | 0.552 | 0.062 | 0.527 | 0.503 |
| trunk/buttock | | | | | | |
| Age 11-20 years | 0.053 | 0.538 | 0.514 | 0.050 | 0.494 | 0.506 |
| Age 21-30 years | 0.045 | 0.549 | 0.495 | 0.042 | 0.466 | 0.464 |
| Lesion onset >12 weeks | 0.041 | 0.503 | 0.487 | 0.035 | 0.470 | 0.477 |
| Lesion onset NI | 0.036 | 0.501 | 0.441 | 0.034 | 0.504 | 0.525 |
| Age 31-40 years | 0.032 | 0.501 | 0.499 | 0.033 | 0.514 | 0.471 |
| Lesion onset ≥6 weeks | 0.032 | 0.476 | 0.516 | 0.029 | 0.513 | 0.526 |
| Digital deformity 100% | 0.031 | 0.486 | 0.479 | 0.030 | 0.519 | 0.487 |
| No digital deformity | 0.026 | 0.519 | 0.480 | | | |
| Digital deformity NI | 0.026 | 0.488 | 0.545 | 0.029 | 0.498 | 0.520 |
| Digital deformity ≥75% | 0.024 | 0.473 | 0.482 | 0.022 | 0.486 | 0.494 |
| Lesion onset 3-6 weeks | 0.024 | 0.468 | 0.435 | 0.029 | 0.512 | 0.504 |
| Digital deformity 25% | 0.023 | 0.493 | 0.505 | 0.035 | 0.510 | 0.541 |
| Lesion onset <3 weeks | 0.019 | 0.491 | 0.512 | | | |
| Age 41-50 years | 0.017 | 0.440 | 0.517 | | | |
| Digital deformity ≥50% | 0.016 | 0.504 | 0.474 | | | |
| Digits amputated | 0.015 | 0.482 | 0.515 | 0.020 | 0.477 | 0.519 |

### Interpreting AI Predictions

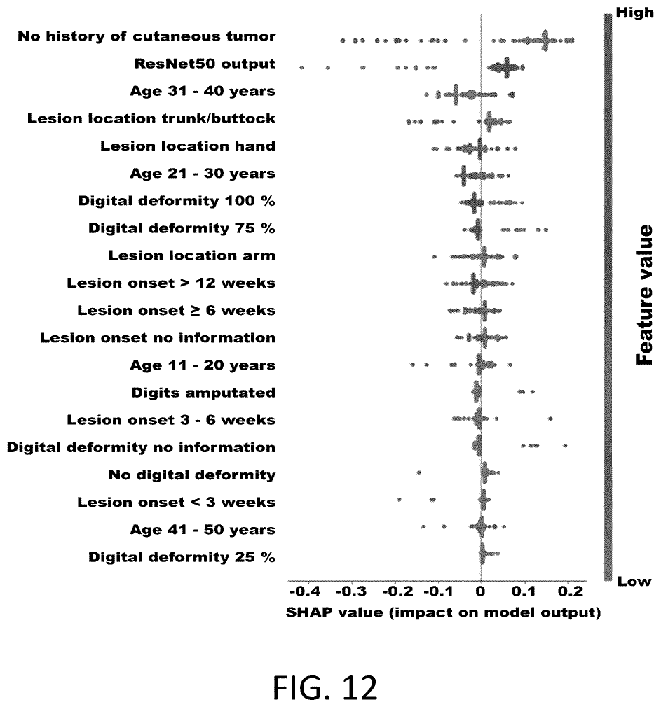

The Mean of Absolute Shapley Additive Explanations (SHAP) Values quantifies the average impact of clinical features on the predictions of a Random Forest model, focusing solely on the magnitude of influence. SHAP values were initially computed for each instance and class (SCC/non-SCC) to assess deviations from the baseline. These values (FIG. 12) were converted to absolute values to highlight their magnitude. Averaging these absolute values provided a class-specific absolute mean SHAP value for each clinical feature, resulting in the overall absolute mean SHAP value (Table 6).

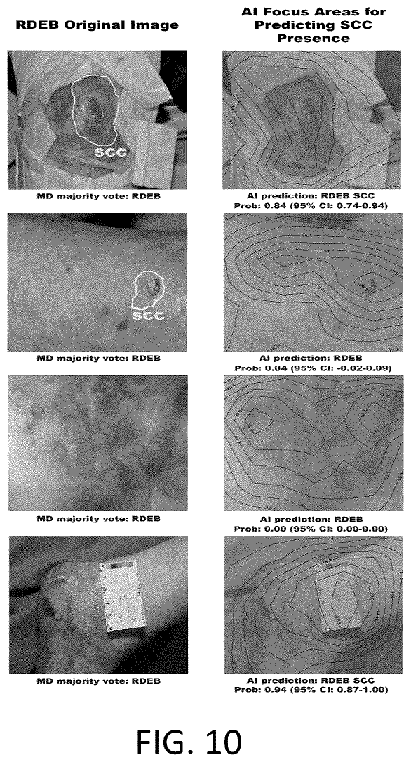

One feature in the Random Forest-based meta-learner is derived from images processed through ResNet50 or ResNet152 models. To identify the specific regions of the image that the model analyzes to detect squamous cell carcinoma (SCC), class activation mapping (CAM) techniques were employed (FIGS. 10 and 13). This process involved passing images through a fully trained neural network, then extracting and weighing feature maps from the last convolutional layer. The composite CAMs were resized to match the dimensions of the original images and overlaid to clearly indicate the key areas significant for diagnosing SCC.

### Clinician Diagnostics

21 clinicians from EB-Clinet and the North American EB Clinical Research Consortium were consented to assess subsets A to E of images using an online REDCap survey. Similar to AI, they received relevant clinical information (Table 4) for each image. Clinicians evaluated the likelihood of SCC in each image. Among the 21 clinicians who evaluated the images, 11 were dermatologists, 8 specialized in pediatric dermatology, and 2 provided healthcare to RDEB patients without a dermatology specialty. Of these clinicians, 66.67% had over 20 years of experience with RDEB, 28.57% had at least 10 years, and one clinician had a minimum of 5 years' experience. Their exposure to SCC varied, with some having treated fewer than 4 cases and others handling over 20 cases of RDEB-SCC. These clinicians practice in 10 different countries with diverse dermatological practices, including Australia, the USA, Canada, the UK, Chile, India, Germany, Switzerland, Austria, and Mexico, reflecting a global spread averaging 4532.595±885.1522 km from the equator.

TABLE 4
Characteristics of Patients in Training and Test Image Sets

| | Images in | | | |
|---|---|---|---|---|
| | Train/validation set | | Test set | |
| | RDEB-non- | RDEB- | RDEB-non- | RDEB- |
| Feature | SCC | SCC | SCC | SCC |
| Total | 282 (100%) | 459 (100%) | 53 (100%) | 128 (100%) |
| Age, years | | | | |
| 11-20 | 64 (22.7%) | 64 (13.9%) | 11 (20.8%) | 15 (11.7%) |
| 21-30 | 107 (37.9%) | 114 (24.8%) | 34 (64.2%) | 80 (62.5%) |
| 31-40 | 78 (27.7%) | 224 (48.8%) | 2 (3.8%) | 17 (13.3%) |
| 41-50 | 15 (5.3%) | 43 (9.4%) | 3 (5.7%) | 15 (11.7%) |
| 51-60 | 18 (6.4%) | 14 (3.1%) | 3 (5.7%) | 1 (0.8%) |
| Sex = Female | 145 (51.4%) | 166 (36.2%) | 21 (39.6%) | 31 (24.2%) |
| Time since lesion onset, | | | | |
| weeks | | | | |
| <3 | 14 (5%) | 4 (0.9%) | 4 (7.5%) | 1 (0.8%) |
| 3-5 | 20 (7.1%) | 50 (10.9%) | 3 (5.7%) | 1 (0.8%) |
| 6-12 | 12 (4.3%) | 19 (4.1%) | 3 (5.7%) | 5 (3.9%) |
| ≥6 | 152 (53.9%) | 234 (51%) | 22 (41.5%) | 83 (64.8%) |
| >12 | 140 (49.6%) | 215 (46.8%) | 19 (35.8%) | 78 (60.9%) |
| NI | 96 (34%) | 171 (37.3%) | 24 (45.3%) | 43 (33.6%) |
| Lesion location | | | | |
| Hand | 41 (14.5%) | 202 (44%) | 0 (0%) | 73 (57%) |
| Foot | 61 (21.6%) | 90 (19.6%) | 10 (18.9%) | 15 (11.7%) |
| Head/neck | 3 (1.1%) | 8 (1.7%) | 1 (1.9%) | 1 (0.8%) |
| Leg | 39 (13.8%) | 66 (14.4%) | 4 (7.5%) | 24 (18.8%) |
| Trunk/buttock | 81 (28.7%) | 26 (5.7%) | 23 (43.4%) | 2 (1.6%) |
| Arm | 57 (20.2%) | 67 (14.6%) | 15 (28.3%) | 13 (10.2%) |
| Digital deformity | | | | |
| Normal hands/no fusion | 16 (5.7%) | 0 (0%) | 0 (0%) | 2 (1.6%) |
| 25% | 13 (4.6%) | 4 (0.9%) | 0 (0%) | 0 (0%) |
| 50% | 2 (0.7%) | 21 (4.6%) | 13 (24.5%) | 0 (0%) |
| 75% | 26 (9.2%) | 7 (1.5%) | 0 (0%) | 1 (0.8%) |
| 100% | 223 (79.1%) | 364 (79.3%) | 40 (75.5%) | 111 (86.7%) |
| Milia and/or scarring | 2 (0.7%) | 0 (0%) | 0 (0%) | 1 (0.8%) |
| Amputated | 0 (0%) | 37 (8.1%) | 0 (0%) | 7 (5.5%) |
| ≥50% | 253 (89.7%) | 429 (93.5%) | 53 (100%) | 120 (93.8%) |
| ≥75% | 251 (89%) | 408 (88.9%) | 40 (75.5%) | 120 (93.8%) |
| NI | 0 (0%) | 26 (5.7%) | 0 (0%) | 6 (4.7%) |
| No history of cutaneous | 194 (68.8%) | 85 (18.5%) | 42 (79.2%) | 4 (3.1%) |
| tumor | | | | |

### Analytical Methods

Primary outcomes measured diagnostic accuracy of meta-learner models, individual clinicians, and dermatologist consensus through sensitivity, specificity, and the area under the curve (AUC) compared to biopsy-confirmed diagnoses. Secondary outcomes assessed diagnostic agreement among clinicians and AI models, using Fleiss Kappa for clinicians and Cohen's Kappa for AI versus clinician consensus. Another secondary outcome assessed the diagnostic accuracy of meta-learner models trained on images with backgrounds removed. All analyses were performed using Python, version 3.9.

### Results

Clinical data and images from patients with RDEB, collected over a specified period, were used to develop an AI diagnostic tool to detect SCC. The data were gathered during routine dermatological care before biopsy. The data was organized into a training set of 741 images and a test set of 181 images (Table 4). The training set had a higher female representation (41.97%) compared to the test set (28.73%), a less right-skewed age distribution, and a lower SCC to non-SCC ratio of 1.63, against 2.42 in the test set. Both sets primarily contained lesions observed for at least six weeks, affecting 52.1% of the training and 58.01% of the test datasets. Documentation on the onset of these lesions was lacking for 36.03% and 37.02% of images, respectively. This significant level of missing data is expected due to the chronic and recurrent nature of RDEB, which often hinders consistent record-keeping. In each set, lesions primarily appeared on the hands (32.8% in the training & 40.33% in the test set) and feet (20.38% in the training & 13.81% in the test set), with the remainder evenly distributed across other body areas. Moreover, a majority of patients in both sets exhibited severe digital deformity (complete webbing), with rates of 79.22% in the training and 83.43% in the test sets. A significant proportion of individuals in both the training (62.35%) and test (74.59%) sets reported a history of cutaneous tumors.

The SCC to non-SCC ratios (1.4±0.09) in sub-sets A to E of the test set (FIG. 11) closely resembled those observed in the training set. Sub-sets A to E underwent evaluation by an international team of 21 clinicians and an AI diagnostic tool.

### Clinician Diagnostic Accuracy

Each clinician was tasked with evaluating images and clinical data from one of subsets A to E. Diagnostic agreement among clinicians, exceeding chance levels as illustrated in Table 2, varied from fair (Fleiss' kappa¹⁹=0.29±0.09 in subsets A, C, D) to moderate (Fleiss' kappa=0.53±0.03 in subsets B and E). Comparative analysis of clinician diagnoses against biopsy results showed a sensitivity of 0.63±0.06, specificity of 0.72±0.2, and an AUC of 0.73±0.08. Clinician performance varied within each sub-set, with individual AUC scores ranging from 0.57 to 0.87. No significant correlations were detected between clinicians' diagnostic accuracy and their specialty or experience with RDEB or SCC, as detailed in Table 5.

TABLE 2
Diagnostic performance and agreement among
clinicians assessing test subsets A to E.

| | | | | Fleiss' |
|---|---|---|---|---|
| Clinician | Sensitivity | Specificity | AUC | Kappa |
| | Sub-set A | | | |
| | (49 images) | | | |
| 1 | 0.53 | 0.5 | 0.57 | 0.39 |
| 2 | 0.61 | 0.9 | 0.7 | |
| 3 | 0.57 | 0.85 | 0.75 | |
| 4 | 0.57 | 0.9 | 0.73 | |
| 5 | 0.65 | 0.85 | 0.64 | |
| Set | 0.59 ± 0.05 | 0.8 ± 0.17 | 0.68 ± 0.07 | |
| Average ± SD | | | | |
| | Sub-set B | | | |
| | (49 images) | | | |
| 1 | 0.57 | 0.9 | 0.73 | 0.55 |
| 2 | 0.67 | 0.75 | 0.77 | |
| 3 | 0.67 | 0.6 | 0.7 | |
| Set | 0.64 ± 0.06 | 0.75 ± 0.15 | 0.73 ± 0.04 | |
| Average ± SD | | | | |
| | Sub-set C | | | |
| | (48 images) | | | |
| 1 | 0.69 | 0.84 | 0.83 | 0.25 |
| 2 | 0.62 | 0.42 | 0.73 | |
| 3 | 0.58 | 0.16 | 0.68 | |
| 4 | 0.65 | 0.84 | 0.78 | |
| Set | 0.64 ± 0.05 | 0.57 ± 0.33 | 0.76 ± 0.06 | |
| Average ± SD | | | | |
| | Sub-set D | | | |
| | (48 images) | | | |
| 1 | 0.71 | 0.85 | 0.83 | 0.22 |
| 2 | 0.62 | 0.4 | 0.69 | |
| 3 | 0.58 | 0.75 | 0.69 | |
| 4 | 0.56 | 0.9 | 0.67 | |
| Set | 0.62 ± 0.07 | 0.73 ± 0.23 | 0.72 ± 0.07 | |
| Average ± SD | | | | |
| | Sub-set E | | | |
| | (30 images) | | | |
| 1 | 0.73 | 0.77 | 0.87 | 0.51 |
| 2 | 0.77 | 0.85 | 0.84 | |
| 3 | 0.6 | 0.62 | 0.69 | |
| 4 | 0.67 | 0.77 | 0.82 | |
| 5 | 0.57 | 0.77 | 0.62 | |
| Set | 0.67 ± 0.08 | 0.76 ± 0.08 | 0.77 ± 0.11 | |
| Average ± SD | | | | |
| Overall | 0.63 ± 0.06 | 0.72 ± 0.2 | 0.73 ± 0.08 | 0.38 ± 0.15 |
| Average ± SD | | | | |

TABLE 5
Clinician counts (%) categorized by professional characteristics.
Statistical significance (p-values) indicates the correlation between
these characteristics and clinicians' performance metrics—sensitivity,
specificity, and AUC—when diagnosing RDEB images.

| | | p-value | | |
|---|---|---|---|---|
| | Clinicians, | Sen- | Spec- | |
| Characteristics | n (%) | sitivity | ificity | AUC |
| Total clinicians | 21 (100%) | | | |
| Specialty | | 0.30 | 0.97 | 0.79 |
| Dermatologist | 11 (52.38%) | | | |
| Pediatric dermatologist | 8 (38.1%) | | | |
| Pediatrician | 1 (4.76%) | | | |
| Other | 1 (4.76%) | | | |
| Years of experience in | | 0.57 | 0.69 | 0.57 |
| treating RDEB patients | | | | |
| 5-10 | 1 (4.76%) | | | |
| 11-20 | 6 (28.57%) | | | |
| >20 | 14 (66.67%) | | | |
| Career total of SCC Cases in | | 0.16 | 0.49 | 0.11 |
| RDEB Patients Observed | | | | |
| 1-3 | 3 (14.29%) | | | |
| 4-5 | 4 (19.05%) | | | |
| 11-20 | 7 (33.33%) | | | |
| >20 | 7 (33.33%) | | | |

### AI Diagnostic Accuracy

Meta-learner models were evaluated on a test set comprising 181 images. The ResNet50-based meta-learner demonstrated superior performance with a sensitivity of 0.93, specificity of 0.70, and an AUC of 0.81. In contrast, the ResNet152-based model achieved a sensitivity of 0.81, specificity of 0.72, and an AUC of 0.76. Among the features, the history of cutaneous tumors (feature importance=0.23) and the output from image processing by ResNet50 (importance=0.13) were the most influential in the decision-making process, both showing a positive correlation with the diagnoses of RDEB-SCC, as indicated by the mean absolute SHAP values (Table 6, FIG. 12). Other significant features impacting the diagnosis of SCC included lesion location on the arm (feature importance=0.07) and hand (feature importance=0.06), along with the patient's age in the ranges of 11-20 years (feature importance=0.05) and 21-30 years (feature importance=0.05). To demonstrate how Residual Networks identify predictive areas within images for carcinoma detection, CAMs were utilized. FIGS. 10 and 13 illustrate the model's focus on regions critical for recognizing skin features associated with the presence or absence of malignancy. These visualizations highlight influential areas without segmenting the image, showcasing the model's diagnostic capabilities.

Two additional meta-learner models were trained on the same dataset without background elements. Both models showed similar diagnostic accuracy (FIG. 13). However, the ResNet152-based classifier demonstrated a higher sensitivity of 0.85, a specificity of 0.74, and an AUC of 0.79. The ResNet50's performance mirrored that of a model trained on original images.

### Diagnostic Accuracy of AI Versus Clinician Consensus

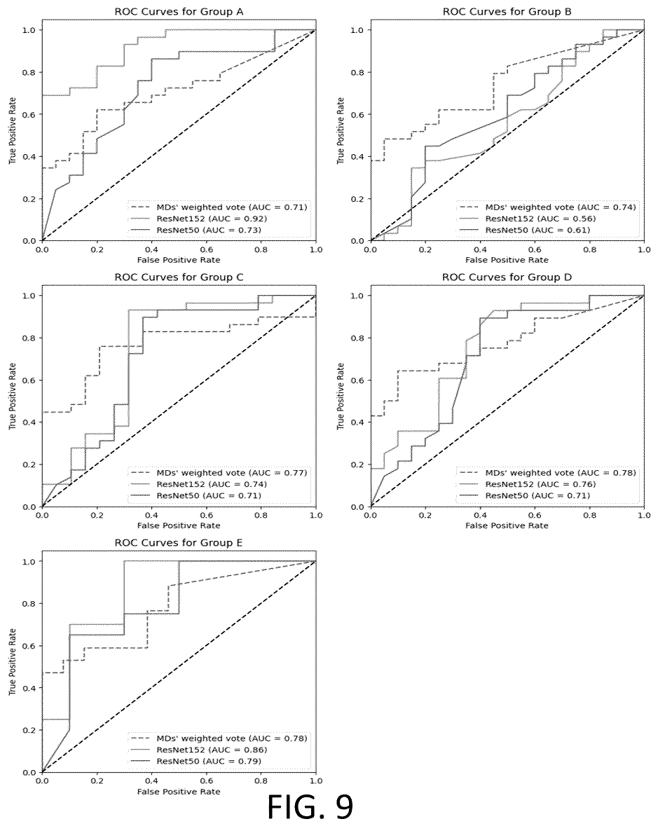

To directly compare clinician and AI diagnostic performances, AI meta-learners were evaluated using subsets A to E, previously assessed by clinicians. The earlier-reported moderate agreement among clinicians led us to use a majority vote approach to derive a consensus diagnosis based on the most common decision for each image. This majority vote was then benchmarked against the performances of meta-learner models (FIG. 9). Variation was noted in the majority vote outcomes across subsets, with an overall sensitivity of 0.51±0.12, specificity of 0.84±0.1, and AUC of 0.70±0.03. The ResNet50-based meta-learner demonstrated comparable performance to the majority clinician vote, achieving a sensitivity of 0.73±0.07, specificity of 0.50±0.06, and AUC of 0.71±0.06. The ResNet152-based meta-learner outperformed the majority clinician vote, with a sensitivity of 0.76±0.12, specificity of 0.60±0.14, and AUC of 0.77±0.14.

TABLE 3
Diagnostic performance of AI models on a test set of 181 images, and AI models
compared with assessments by international clinicians on test subsets A-E.

| | Clinician's weighted vote | | | ResNet50 | | | Resnet152 | | |
|---|---|---|---|---|---|---|---|---|---|
| Data Set | Sensitivity | Specificity | AUC | Sensitivity | Specificity | AUC | Sensitivity | Specificity | AUC |

Complete test set (181 image)

| | | | | | | |
|---|---|---|---|---|---|---|
| with background | 0.93 | 0.7 | 0.81 | 0.81 | 0.72 | 0.76 |
| without background | 0.93 | 0.7 | 0.81 | 0.85 | 0.74 | 0.79 |

Subsets of the test set with background

| | | | | | | | | | |
|---|---|---|---|---|---|---|---|---|---|
| set A (49 images) | 0.34 | 0.95 | 0.69 | 0.71 | 0.5 | 0.73 | 0.84 | 0.65 | 0.92 |
| set B (49 images) | 0.55 | 0.75 | 0.74 | 0.63 | 0.4 | 0.61 | 0.55 | 0.35 | 0.56 |
| set C (48 images) | 0.62 | 0.79 | 0.74 | 0.77 | 0.58 | 0.71 | 0.81 | 0.68 | 0.74 |
| set D (48 images) | 0.43 | 0.95 | 0.74 | 0.73 | 0.5 | 0.71 | 0.75 | 0.6 | 0.76 |
| set E (30 images) | 0.59 | 0.77 | 0.77 | 0.83 | 0.5 | 0.79 | 0.83 | 0.7 | 0.86 |
| Overall average ± SD | 0.51 ± | 0.84 ± | 0.7 ± | 0.73 ± | 0.5 ± | 0.71 ± | 0.76 ± | 0.6 ± | 0.77 ± |
| | 0.12 | 0.1 | 0.03 | 0.07 | 0.06 | 0.06 | 0.12 | 0.14 | 0.14 |

### Example 3

The dataset comprised multiple sets of images, with each set depicting the same lesion from different viewpoints or similar lesions on the same individual. To maintain data integrity, it was manually ensured that the sets were not divided between the training and test sets. Notably, there was no overlap between the images in the test and the training sets in terms of identical lesions with multiple viewpoints. A total of 924 professional images, comprising 587 EB SCC and 361 EB non-SCC, were allocated into distinct datasets for model training and validation. Specifically, the dataset was divided into a training set of 745 images (459 EB SCC and 282 EB non-SCC) and a test set of 181 images (128 EB SCC and 53 EB non-SCC), representing approximately 80% and 20% of the total dataset, respectively.

A diverse array of models were evaluated, encompassing both convolutional neural networks (CNNs) and a traditional machine learning algorithm (Random Forest), to address our research objectives. The CNN architectures examined include: (i) ResNet50, ResNet101, and ResNet152, which are part of the Residual Networks family known for their deep architectures facilitated by skip connections that mitigate the vanishing gradient problem; (ii) Xception, which leverages depth wise separable convolutions to enhance model efficiency and performance; (iii) VGG16, which is characterized by its simplicity and depth, employing consecutive 3×3 convolutional layers; and (iv) DenseNet121, which is distinct for its dense connectivity pattern, ensuring maximum information flow between layers in the network. Beyond CNNs, we explored the Vision Transformer (ViT), which applies the transformer architecture to image analysis, capturing global dependencies within the image. Lastly, the Random Forest model, a traditional machine learning algorithm, uses an ensemble of decision trees to reduce overfitting and improve prediction accuracy. Each model was selected for its unique architectural features or learning strategy, offering a comprehensive overview of current methodologies in image classification and analysis.

To enhance the robustness and generalizability of the model, a 5-fold cross-validation method was employed. This approach involved partitioning the training dataset into five equal-sized subsets. In each cross-validation cycle, four subsets were used for training the model, while the remaining subset served as the validation set to assess model performance. This process was repeated five times, with each subset used exactly once as the validation set, ensuring comprehensive evaluation and utilization of the data for model optimization.

Data augmentation was applied in all model training processes, with the exception of the RandomForest model. Data augmentation, a technique to artificially expand the diversity of a dataset by applying various transformations to the original images, can significantly impact the performance of deep learning models such as ResNet50, ResNet101, ResNet152, ViT, VGG16, and DenseNet121. By introducing variations such as rotations, flips, scaling, and color adjustments, data augmentation can enhance the models' ability to generalize from the training data to new, unseen data, potentially improving accuracy and robustness against overfitting. For CNNs like ResNet variants and VGG16, data augmentation exploits spatial hierarchies and invariances, making these models more adept at recognizing patterns and features under varied conditions. Similarly, for architectures like ViT that rely on global context and attention mechanisms, data augmentation can provide a richer set of contextual scenarios, promoting better feature extraction and classification performance. DenseNet121, known for its efficient feature reuse, can also benefit from augmented datasets by learning more diverse representations. However, the effectiveness of data augmentation is contingent on the relevance and diversity of the transformations applied, necessitating careful selection to match the specific challenges and characteristics of the task at hand.

The optimal model was determined through a comprehensive evaluation of its performance, utilizing a suite of statistical measures to ensure a balanced assessment across various aspects of predictive accuracy and reliability. Specifically, the model's effectiveness was gauged using Accuracy, which measures the overall correctness of predictions; Precision and Positive Predictive Value (PPV), which assess the model's ability to accurately predict positive instances; Recall/Sensitivity, indicating the model's capacity to identify all actual positive cases; F1 score, a harmonic mean of Precision and Recall, providing a single metric to balance the trade-off between them; Specificity, evaluating the model's ability to correctly identify negative instances; and Negative Predictive Value (NPV), which measures the accuracy of predicting negative outcomes. This multifaceted approach ensured that the selected model not only excels in identifying true positives and negatives but also maintains a low rate of false positives and negatives, embodying a robust predictive tool suitable for practical application.

Among the fine-tuned convolutional neural network (CNN) models, ResNet50 and ResNet152, alongside VGG16 and DenseNet121, demonstrated (Table 7) superior performance, achieving F1 scores of 0.84, 0.84, 0.77, and 0.71, respectively. Further enhancement was observed when patient characteristics were integrated with the models. Meta-learner models based on ResNet50 and VGG16 emerged as the most effective, with F1 scores of 0.90 and 0.87, correspondingly.

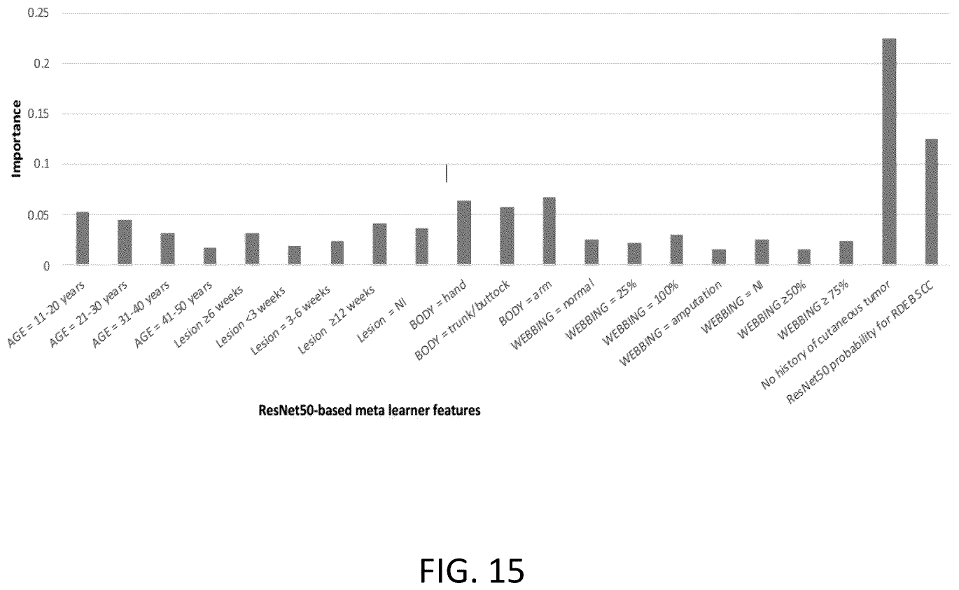

Feature importance serves as a crucial metric for evaluating the contribution of each variable to the model's overall prediction accuracy. This ensemble method, comprising multiple decision trees, assesses feature importance by averaging the decrease in impurity—such as Gini impurity for classification tasks or variance for regression—attributed to each feature across all trees. Generally, a feature that significantly reduces impurity is deemed more critical for the model's performance. While most features exhibited importance scores around 0.05 (FIG. 15), indicating a modest impact on predictive accuracy, two features stood out for their substantial contributions: The prediction from the CNN emerged with an importance score of 0.13, and the history of cutaneous tumor was even more influential, with a score of 0.22. These findings underscore the relative significance of these two features in enhancing the model's ability to make accurate predictions, highlighting their pivotal roles in the predictive framework of our Random Forest model.

TABLE 7
Performance of classifier models measured using various statistical metrics.

| Classifier | Performance Metrics | | | | | | | | | | |
|---|---|---|---|---|---|---|---|---|---|---|---|
| model | TN | FP | FN | TP | Acuracy | Precision | Recall Sensitivity | F1 | Specificity | PPV | NPV |

Tuned-up models using RDEB images with and without SCC

| | | | | | | | | | | | |
|---|---|---|---|---|---|---|---|---|---|---|---|
| ResNet50 | 24 | 29 | 15 | 113 | 0.76 | 0.80 | 0.88 | 0.84 | 0.45 | 0.80 | 0.62 |
| VGG16 | 24 | 29 | 29 | 99 | 0.68 | 0.77 | 0.77 | 0.77 | 0.45 | 0.77 | 0.45 |
| ResNet152 | 24 | 29 | 14 | 114 | 0.76 | 0.80 | 0.89 | 0.84 | 0.45 | 0.80 | 0.63 |
| DenseNet121 | 25 | 28 | 43 | 85 | 0.61 | 0.75 | 0.66 | 0.71 | 0.47 | 0.75 | 0.37 |

Models trained with the use of patient's characteristics only

| | | | | | | | | | | | |
|---|---|---|---|---|---|---|---|---|---|---|---|
| Random | 32 | 21 | 5 | 123 | 0.86 | 0.85 | 0.95 | 0.90 | 0.60 | 0.85 | 0.86 |
| Forest | | | | | | | | | | | |

Random Forest-based meta learners based on patient's characteristics and Input from tunned-up models

| | | | | | | | | | | | |
|---|---|---|---|---|---|---|---|---|---|---|---|
| ResNet50 | 37 | 16 | 9 | 119 | 0.86 | 0.88 | 0.93 | 0.90 | 0.70 | 0.88 | 0.80 |
| VGG16 | 37 | 16 | 17 | 111 | 0.82 | 0.87 | 0.87 | 0.87 | 0.70 | 0.87 | 0.69 |
| ResNet152 | 38 | 15 | 24 | 104 | 0.78 | 0.87 | 0.81 | 0.84 | 0.72 | 0.87 | 0.61 |
| DenseNet121 | 39 | 14 | 28 | 100 | 0.77 | 0.88 | 0.78 | 0.83 | 0.74 | 0.88 | 0.58 |

### REFERNCES

The following references are herein incorporated by reference in their entirety.
1. Has C, Bauer J W, Bodemer C, et al. Consensus reclassification of inherited epidermolysis bullosa and other disorders with skin fragility. Br J Dermatol. October 2020; 183(4):614-627.doi:10.1111/bjd.18921
2. Has C, Hess M, Anemüller W, et al. Epidemiology of inherited epidermolysis bullosa in Germany. Journal of the European Academy of Dermatology and Venereology. 2023; 37(2):402-410.doi:https://doi.org/10.1111/jdv.18637
3. Fine J D, Johnson L B, Weiner M, Li K P, Suchindran C. Epidermolysis bullosa and the risk of life-threatening cancers: the National E B Registry experience, 1986-2006. J Am Acad Dermatol. February 2009; 60(2):203-11.doi:10.1016/j.jaad.2008.09.035
4. Mellerio J E, Robertson S J, Bernardis C, et al. Management of cutaneous squamous cell carcinoma in patients with epidermolysis bullosa: best clinical practice guidelines. British Journal of Dermatology. 2016; 174(1):56-67. doi:https://doi.org/10.1111/bjd.14104
5. Liu X, Cruz Rivera S, Moher D, et al. Reporting guidelines for clinical trial reports for interventions involving artificial intelligence: the CONSORT-AI extension. Nature Medicine. 2020 Sep. 1 2020; 26(9):1364-1374.doi:10.1038/s41591-020-1034-x
6. Esteva A, Kuprel B, Novoa R A, et al. Dermatologist-level classification of skin cancer with deep neural networks. Nature. 2017 Feb. 1 2017; 542(7639):115-118. doi:10.1038/nature21056
7. Haenssle H A, Fink C, Schneiderbauer R, et al. Man against machine: diagnostic performance of a deep learning convolutional neural network for dermoscopic melanoma recognition in comparison to 58 dermatologists. Ann Oncol. Aug. 1 2018; 29(8):1836-1842.doi:10.1093/annonc/mdy166
8. Tschandl P, Rinner C, Apalla Z, et al. Human-computer collaboration for skin cancer recognition. Nature Medicine. 2020 Aug. 1 2020; 26(8):1229-1234. doi:10.1038/s41591-020-0942-0
9. Schaefer J, Lehne M, Schepers J, Prasser F, Thun S. The use of machine learning in rare diseases: a scoping review. Orphanet Journal of Rare Diseases. 2020 Jun. 9 2020; 15(1):145.doi:10.1186/s13023-020-01424-6
10. Issa C J, Reimer-Taschenbrecker A, Paller A S. A call for implementing augmented intelligence in pediatric dermatology. Pediatric Dermatology. 2023; 40(3):584-586. doi:https://doi.org/10.1111/pde.15298
11. Affairs UNOftCoH. (2024 January). 2024 Humanitarian Needs and Response Plan for Ukraine. Accessed Aug. 16, 2024. https://www.unhcr.org/ua/wp-content/uploads/sites/38/2024/01/Ukraine-HNRP-2024-Humanitarian-Needs-and-Response-Plan-UA-20240110.pdf
12. Collins G S, Moons K G M, Dhiman P, et al. TRIPOD+AI statement: updated guidance for reporting clinical prediction models that use regression or machine learning methods. BMJ. 2024; 385: e078378.doi:10.1136/bmj-2023-078378
13. Kirillov A, Mintun E, Ravi N, et al. Segment Anything. arXiv: 230402643. 2023;
14. Zhou B, Khosla A, Lapedriza A, Oliva A, Torralba A. Learning Deep Features for Discriminative Localization. 2016:2921-2929.
15. Deng J, Dong W, Socher R, Li L J, Li K, Fei-Fei L. ImageNet: A large-scale hierarchical image database. 2009:248-303.
16. Breiman L. Random Forests. Machine Learning. 2001 Oct. 1 2001; 45(1):5-32. doi:10.1023/A: 1010933404324
17. Cohen J. A Coefficient of Agreement for Nominal Scales. Educational and Psychological Measurement. 1960; 20(1):37-46.doi:10.1177/001316446002000104
18. Python Software Foundation. (2020). Python language reference, version 3.9. Accessed Aug. 16, 2024. https://www.python.org
19. Fleiss J L. Measuring nominal scale agreement among many raters. Psychological Bulletin. 1971; 76(5):378-382.doi:10.1037/h0031619
20. Adachi T, El-Hattab A W, Jain R, et al. Enhancing Equitable Access to Rare Disease Diagnosis and Treatment around the World: A Review of Evidence, Policies, and Challenges. Int J Environ Res Public Health. Mar. 8 2023; 20(6)doi:10.3390/ijerph20064732
21. Lee J, Liu C, Kim J, et al. Deep learning for rare disease: A scoping review. J Biomed Inform. November 2022; 135:104227.doi:10.1016/j.jbi.2022.104227
22. Faviez C, Chen X, Garcelon N, et al. Diagnosis support systems for rare diseases: a scoping review. Orphanet Journal of Rare Diseases. 2020 Apr. 16 2020; 15(1):94. doi:10.1186/s13023-020-01374-z
23. Zerka F, Urovi V, Bottari F, et al. Privacy preserving distributed learning classifiers-Sequential learning with small sets of data. Computers in Biology and Medicine. 2021 Sep. 1, 2021; 136:104716. doi:https://doi.org/10.1016/j.compbiomed.2021.104716
24. Hasani N, Farhadi F, Morris M A, et al. Artificial Intelligence in Medical Imaging and its Impact on the Rare Disease Community: Threats, Challenges and Opportunities. PET Clin. January 2022; 17(1):13-29.doi:10.1016/j.cpet.2021.09.009

1. A method of developing an artificial intelligence (AI) tool for distinguishing between two medical conditions, the method comprising:
(a) training the AI tool on a large-scale hierarchical image database to generate an initially trained model with robust non-specific feature-detection capabilities;
(b) adapting the initially trained model to the medical conditions by transfer learning to binary classification of images of the two medical conditions to generate model trained on the two medical conditions;
(c) combining output of the del trained on the two medical conditions with patient clinical data to generate a combined model; and
(d) training a Random Forest-based meta-learner classifier with the combined model to generate the AI tool for distinguishing between two medical conditions.

2. The method of claim 1, wherein the large-scale hierarchical image database is not specific to images of the two medical conditions.

3. The method of claim 1, further comprising applying augmentation techniques to expand diversity of the images of the two medical conditions used to trail the AI tool.

4. The method of claim 3, where augmentation techniques comprise transformations of the images of the two medical conditions used to trail the AI tool.

5. The method of claim 4, wherein transformations of the images comprise one or more of rotations, flips, scaling, and color adjustments.

6. The method of claim 1, wherein the two medical conditions are skin conditions.

7. The method of claim 1, wherein the two medical conditions can be differentiated visually.

8. The method of claim 1, wherein the two medical conditions are (1) squamous cell carcinoma (SCC) lesion in a subject suffering from epidermolysis bullosa and (2) non-SCC lesion in a subject suffering from epidermolysis bullosa.

9. The method of claim 8, wherein the subject suffers from recessive dystrophic epidermolysis bullosa (RDEB).

10. A method of determining whether a subject suffers from a first or second medical condition, the method comprising:
(a) obtaining an image of a region of the subject displaying a symptom of the medical condition;
(b) providing the image to an AI tool developed by the method of claim 1;
(c) determining whether the subject suffers from the first or second condition.

11. A method of developing an artificial intelligence (AI) tool for distinguishing between (1) a squamous cell carcinoma (SCC) lesion in a subject suffering from epidermolysis bullosa and (2) a non-SCC lesion in a subject suffering from epidermolysis bullosa:
(a) training the AI tool on a large-scale hierarchical image database not limited to SCC lesions and/or subjects suffering from EB to generate an initially trained model with robust non-specific feature-detection capabilities;
(b) adapting the initially trained model to the medical conditions by transfer learning to binary classification of images of SCC lesions and non-SCC lesions in subject suffering from EB to generate specific model trained on SCC lesions and non-SCC lesions in subject suffering from EB;
(c) combining output of the specific model with patient clinical data to generate a combined model; and
(d) training a Random Forest-based meta-learner classifier with the combined model to generate the AI tool for distinguishing between SCC lesions and non-SCC lesions in subject suffering from EB.

12. The method of claim 11, wherein the subject suffers from recessive dystrophic epidermolysis bullosa (RDEB).

13. A method of distinguishing between an SCC lesion and a non-SCC lesion in subject suffering from EB, the method comprising:
(a) obtaining an image of the lesion;
(b) providing the image to an AI tool developed by the method of claim 11;
(c) determining whether the subject suffers from an SCC lesion or a non-SCC lesion.

14. The method of claim 13, further comprising treating the subject for an SCC lesion or a non-SCC lesion based on the outcome of step (c).

15. A method of determining whether a lesion on a subject suffering from epidermolysis bullosa (EB) is a squamous cell carcinoma (SCC) lesion or a non-SCC lesion, the method comprising:
(a) obtaining an image of the lesion;
(b) providing the image to an AI tool trained to distinguish between SCC lesions and non-SCC lesions on a subject suffering from EB; and
(c) determining whether the lesion is an SCC lesion of a non-SCC lesion.

16. The method of claim 11, further comprising administering appropriate treatment for an SCC lesion or non-SCC lesion.
